(19) United States
(12) Patent Application Publication
(10) Pub. No.: US 2026/0083448 A1
(43) Pub. Date: Mar. 26, 2026

(54) SUTURE/ANCHOR AND DELIVERY DEVICE COMBINATION

(71) Applicants: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(72) Inventor: Mark Edwin HOUSMAN, North Attleboro, MA (US)

(21) Appl. No.: 19/108,785

(22) PCT Filed: Sep. 20, 2023
(86) PCT No.: PCT/US2023/033267

§ 371 Date: Mar. 5, 2025

Related U.S. Application Data
(60) Provisional application No. 63/409,965, filed on Sep. 26, 2022.

(51) Int. Cl. A61B 17/04; A61F 2/08

(52) U.S. Cl. CPC A61B 17/0401; A61F 2/0811; A61B 2017/0409; A61B 2017/0464; A61F 2002/0852; A61F 2002/0882

(57) ABSTRACT

A suture/anchor and delivery device combination includes a first cannulated suture and a second stepped or staged suture that has a smaller diameter section connected to a larger diameter section. The smaller diameter of the second suture is passed through the first suture and the assemblage of sutures is further passed through a tube which acts as an inserter. To actuate the device, the larger diameter of the second suture is pulled into the lumen of the first suture, thereby expanding the first diameter of the first suture to create an interference fixation within bone.

### FIELD

The present disclosure relates to surgical repair of a joint and, more specifically, to devices and methods for inserting a suture anchor used in such a repair.

### BACKGROUND

Anterior shoulder dislocations are common injuries resulting, for example, from falls or overhead throwing. These dislocations can result in pain, apprehension during range of motion, and increased risk of recurrent dislocations. Such recurrent dislocations often result in a “Hill-Sachs defect,” a damaged area of bone and cartilage on the humeral head, which can result in further pain, dislocation, and shoulder instability.

One current surgical solution for reducing the risk of recurrent anterior shoulder dislocations is a Laterjet procedure, in which a bone block is fixed to the anterior side of the glenoid to increase the glenoid surface area and reduce the risk of the humerus falling out of the glenoid socket. Another procedure is capsular tightening, in which the shoulder joint is surrounded by a capsule. When this capsule is tightened with sutures, there is less room within the joint space, reducing the risk of dislocation.

Current surgical procedures to address the Hill-Sachs defect include humeral head grafting, in which a bone graft is placed in the Hill-Sachs defect area. Another procedure is remplissage, in which infraspinatus rotator cuff tissue is pulled into the Hill-Sachs defect with a suture anchor. Yet another procedure is disimpaction, in which the underside of the Hill-Sachs defect is filled with a bone simulant or bone cement. A final procedure is prothesis, which includes a full or partial shoulder replacement.

Each of the above procedures generally requires placement of large, hard anchors in bone that can create additional damage if the anchor inadvertently becomes dislodged from the repair. Therefore, it would be advantageous to provide a smaller, softer anchor to avoid additional damage to the repair, while also improving the fixation strength of the anchor.

### SUMMARY

This disclosure describes a suture/anchor and delivery device combination that includes a micro size, all-suture anchor that can be used to reattach the labrum to the glenoid in a shoulder dislocation repair. A micro size anchor is generally defined as an anchor having a diameter smaller than 1 mm. The device includes a first cannulated suture and a second stepped or staged suture that has a smaller diameter section connected to a larger diameter section. The smaller diameter of the second suture is passed through the first suture and the assemblage of sutures is further passed through an elongated tube which acts as an inserter. To actuate the device, the larger diameter of the second suture is pulled into the lumen of the first suture, thereby expanding the first diameter of the first suture. This increased diameter creates an interference fixation with bone. The soft suture anchor advantageously reduces the risk of additional damage that can occur if the anchor becomes inadvertently dislodged from the repair. Furthermore, the soft anchor allows for superior fixation compared with hard anchors of similar size due to expansion of the all-suture anchor.

Further examples of the suture/anchor and delivery device combination of this disclosure may include one or more of the following, in any suitable combination.

Examples of a suture/anchor delivery and device combination of this disclosure include a tube having a distal end and a proximal end. The tube defines a channel extending from the distal end to the proximal end. A first flexible strand has a distal end and a proximal end. The first flexible strand extends through the channel of the tube such that the distal end of the first flexible strand extends from the distal end of the tube. A second flexible strand has a first region with a first diameter and a second region with a second diameter. The first diameter is selected to be smaller than the second diameter. The first region of the second flexible strand extends through a lumen defined by the first flexible strand such that the second region is adjacent the distal end of the first flexible strand. The second region of the second flexible strand is configured to be pulled into the lumen of the first flexible strand to expand a diameter of the first flexible strand.

In further examples, the tube comprises a rigid or semi-rigid material. In examples, the first flexible strand comprises a hollow monofilament. In examples, the second flexible strand comprises one of mono-filament, muti-filament, braid or mesh. In examples, a transition region extends between the first region and the second region of the second flexible strand. In examples, the first flexible strand is configured to expand to about 0.5 mm. In examples, a first region of the second flexible strand comprises a 2-0) suture. In examples, a second region of the second flexible strand comprises a No. 2 suture. In examples, a size of the second region of the second flexible strand is selected such that the second flexible strand can slide through the lumen of the first flexible strand without substantial interference. In examples, a size of the first flexible strand is selected such that it forms a friction fit with the channel of the tube. In examples, the first flexible strand extends through the channel of the tube such that the proximal end of the first flexible strand extends from the proximal end of the tube. In examples, the first region of the second flexible strand extends through the lumen defined by the first flexible strand such that the first region extends from the proximal end of the first flexible strand.

Examples of a method of surgical repair of this disclosure include inserting a suture/anchor delivery device combination into a hole in bone. The device includes a tube having a distal end and a proximal end. The tube defines a channel extending from the distal end to the proximal end. A first flexible strand has a distal end and a proximal end. The first flexible strand extends through the channel of the tube such that the distal end of the first flexible strand extends from the distal end of the tube. A second flexible strand has a first region with a first diameter and a second region with a second diameter. The first diameter is selected to be smaller than the second diameter. The first region of the second flexible strand extends through a lumen defined by the first flexible strand such that the second region is adjacent the distal end of the first flexible strand. The method also includes pulling the second region of the second flexible strand into the lumen of the first flexible strand to expand a diameter of the first flexible strand.

In further examples, the expanded diameter of the first flexible strand is larger than a diameter of the channel of the tube. In examples, the expanded diameter of the first flexible strand forms an interference fit with the hole. In examples, the method also includes drilling the hole in the bone. In examples, the bone is a glenoid. In examples, the method also includes, after expanding the diameter of the first flexible strand, removing the tube from the hole. In examples, the first flexible strand extends through the channel of the tube such that the proximal end of the first flexible strand extends from the proximal end of the tube. In examples, the first region of the second flexible strand extends through the lumen defined by the first flexible strand such that the first region extends from the proximal end of the first flexible strand.

A reading of the following detailed description and a review of the associated drawings will make apparent the advantages of these and other features. Both the foregoing general description and the following detailed description serve as an explanation only and do not restrict aspects of the disclosure as claimed.

### BRIEF DESCRIPTION OF THE DRAWINGS

Reference to the detailed description, combined with the following figures, will make the disclosure more fully understood, wherein:

FIGS. 1A and 1B illustrate a shoulder joint during a shoulder dislocation:

FIG. 1C illustrates a Hill-Sachs defect resulting from frequent dislocations of the shoulder joint:

FIG. 2A illustrates an example of a suture/anchor and delivery device combination of this disclosure in a disassembled view:

FIG. 2B is a detailed view of an example of the first flexible strand of the device of this disclosure:

FIG. 2C is a detailed view of a portion of the second flexible strand of the device of this disclosure:

FIG. 2D illustrates an example of the device of this disclosure in an assembled view:

FIG. 3A illustrates an example of a deployment of the device of this disclosure in a surgical repair:

FIG. 3B shows an example of the deployed device of this disclosure in a disassembled state:

FIG. 3C is a detailed view of the distal end of the first flexible member of this disclosure; and

FIGS. 4A-E illustrate an example of the use of the device of this disclosure in a surgical repair.

### DETAILED DESCRIPTION

In the following description, like components have the same reference numerals, regardless of different illustrated examples. To illustrate examples clearly and concisely, the drawings may not necessarily reflect appropriate scale and may have certain features shown in somewhat schematic form. The disclosure may describe and/or illustrate features in one example, and in the same way or in a similar way in one or more other examples, and/or combined with or instead of the features of the other examples.

In the specification and claims, for the purposes of describing and defining the invention, the terms “about” and “substantially” represent the inherent degree of uncertainty attributed to any quantitative comparison, value, measurement, or other representation. The terms “about” and “substantially” moreover represent the degree by which a quantitative representation may vary from a stated reference without resulting in a change in the basic function of the subject matter at issue. Open-ended terms, such as “comprise,” “include,” and/or plural forms of each, include the listed parts and can include additional parts not listed, while terms such as “and/or” include one or more of the listed parts and combinations of the listed parts.

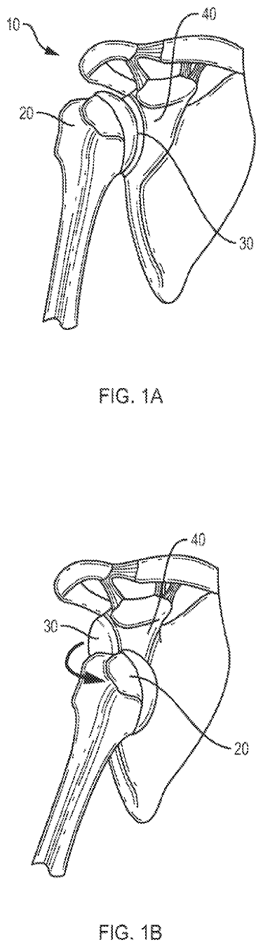

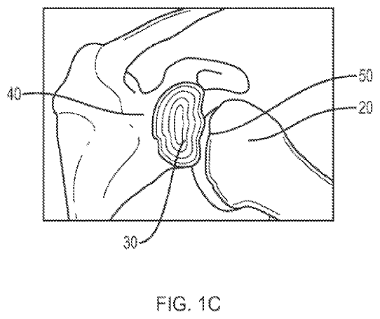

FIGS. 1A and 1B illustrate a dislocation of a shoulder joint. As shown in FIG. 1A, in a normal anatomy, the humeral head 20 resides within the labrum 30, a cup-shaped rim of cartilage that lines the socket of the glenoid 40 and reinforces the ball-and-socket shoulder joint 10. As shown in FIG. 1B, when the shoulder joint 10 is dislocated, the humeral head 20 becomes dislodged from the labrum 30. FIG. 1C illustrates a Hill-Sachs defect 50 resulting from frequent dislocations of the shoulder joint 10.

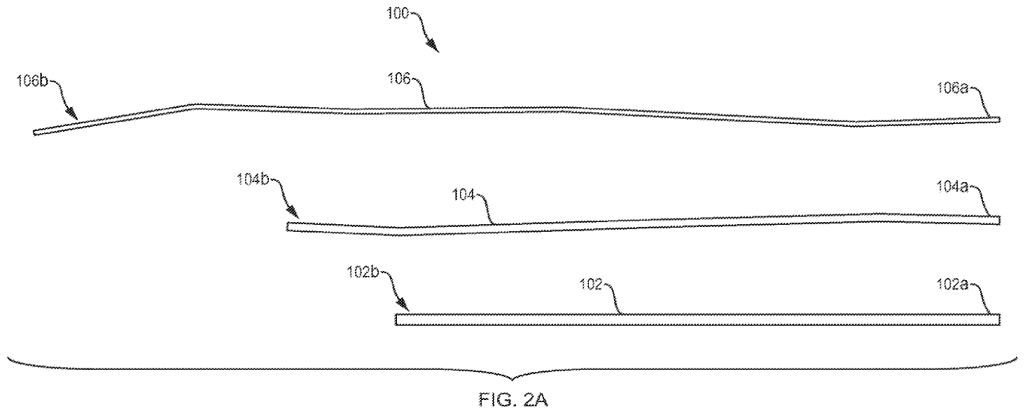

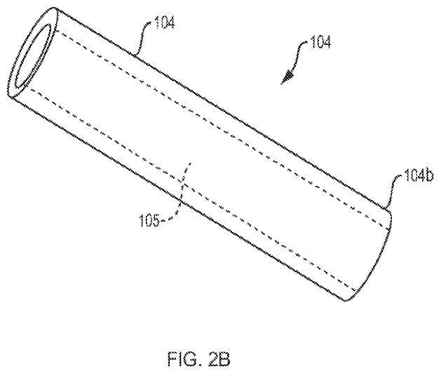

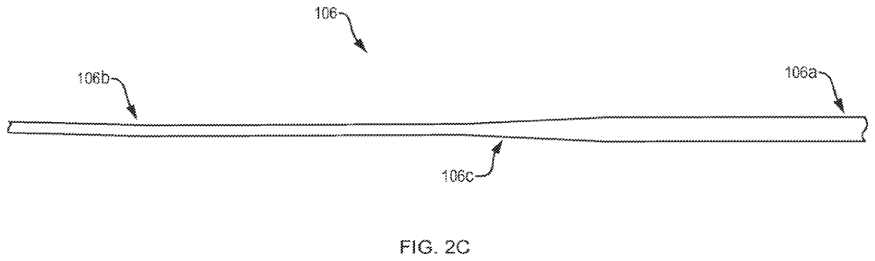

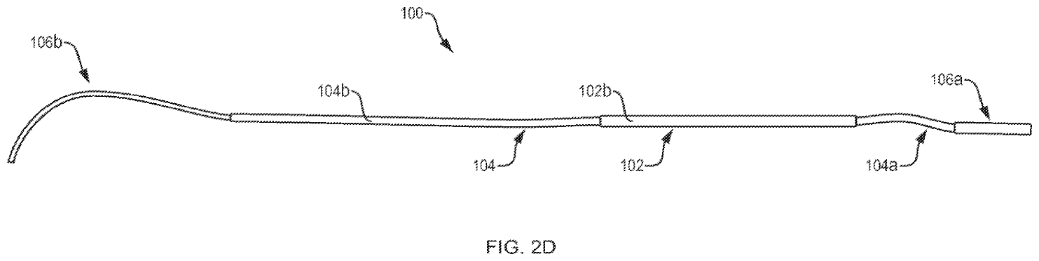

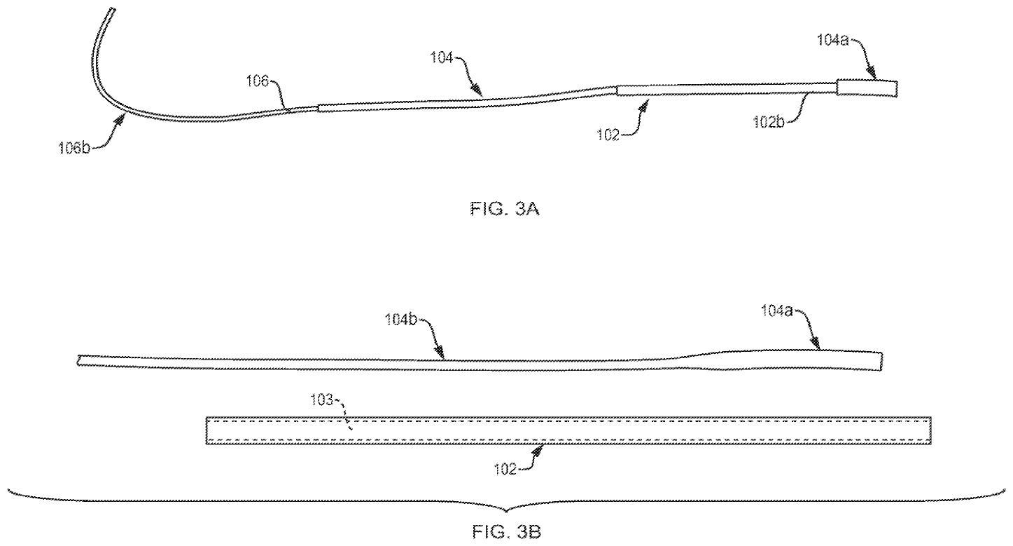

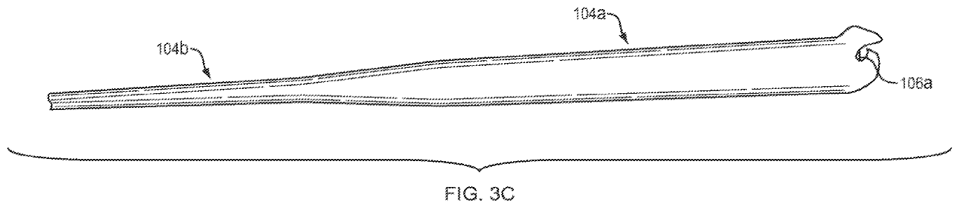

FIG. 2A illustrates an example of a suture/anchor delivery device 100 of this disclosure in a disassembled view. As shown in FIG. 2A, the device 100 may generally comprise an elongated tube 102, a first flexible strand 104, and a second flexible strand 106. The tube 102 may have a distal end 102a and a proximal end 102b. The tube 102 may define a channel 103 (FIG. 3B) extending from the distal end 102a to the proximal end 102b. In examples, the tube 102 may be made from rigid or semi rigid materials, such as hard plastics or metals. The first flexible strand 104 may have a distal end 104a and a proximal end 104b. The first flexible strand 104 may further define a lumen 105 (FIG. 2B) extending from the distal end 104a to the proximal end 104b. In examples, the first flexible strand 104 may be made from a hollow, flexible material, such as a monofilament. The second flexible strand 106 may have a distal end 106a and a proximal end 106b. In examples, the second flexible strand 106 may be made from a flexible material, such as a mono- or muti-filament, braid or mesh. The disclosure contemplates that the second flexible strand may be cannulated or not cannulated.

FIG. 2C is a detailed view of a portion of the second flexible strand 106. As shown in FIG. 2C, a diameter of the distal end 106a of the second flexible strand 106 may be selected to be greater than a diameter of the proximal end 106b. A transition region 106c may extend between the distal end 106a and the proximal end 106b of the flexible strand 106. A size of both the distal end 106a and the proximal end 106b of the second flexible strand 106 may be selected such that the second flexible strand 106 can slide through the lumen 105 of the first flexible strand 104 without substantial interference. In examples, the proximal end 106b of second flexible strand 106 may transition from a 2-0) suture to a No. 2 suture at the distal end 106a.

FIG. 2D illustrates an example of the device 100 of this disclosure in an assembled view: As shown in FIG. 2D, before deployment, the second flexible strand 106 may extend through the lumen 105 of the first flexible strand 104 such that the distal end 106a of the second flexible strand 106 is adjacent the distal end 104a of the first flexible strand 104 and proximal end 106b of the second flexible strand 106 extends from the proximal end 104b of the first flexible strand 104. Both the first flexible strand 104 and the second flexible strand 106 may further extend through the tube 102 such that the distal end 104a of the first flexible strand 104 and the distal end 106a of the second flexible strand 106 extend from the distal end 102a of the tube 102. The proximal end 104b of the first flexible strand 104 and the proximal end 106b of the second flexible strand 106 may also extend from the proximal end 102b of the tube 102. A size of the first flexible strand 104 may be selected such that it forms a friction fit with the channel 103 of the tube 102.

FIG. 3A illustrates an example of the deployment of the device 100 in a surgical repair. As shown in FIG. 3A, to deploy the device 100, the user may pull the proximal end 106b of the second flexible strand 106 proximally such that the distal end 106a of the second flexible strand 106 enters the lumen 105 of the first flexible strand 104, causing expansion of the distal end 104a of the first flexible strand 104 against the distal end 102a of the tube. When expanded, the distal end 104a of the first flexible strand 104 may have a diameter that is larger than a diameter of the channel 103 of the tube 102 such that the expanded distal end 104a cannot be pulled proximally through the tube 102. For example, a diameter of the expanded distal end 104a may be about 0.5 mm. Because of the extended length of the tube 102 and the friction fit between the tube 102 and the first flexible member 104, the first flexible member 104 remains substantially stationary within the tube 102 during deployment. FIG. 3B shows an example of the deployed device 100 in a disassembled state, including the expanded distal end 104a of the first flexible member relative to the unexpanded proximal end 104b, and the tube 102 including the channel 103. FIG. 3C shows a detailed view of the expanded distal end 104a of the first flexible member 104 relative to the non-expanded proximal end 104b.

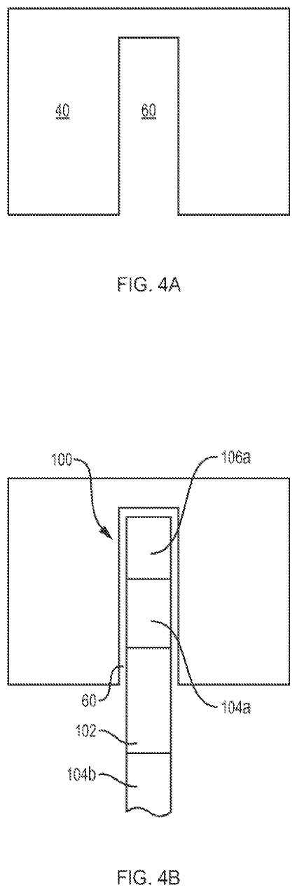

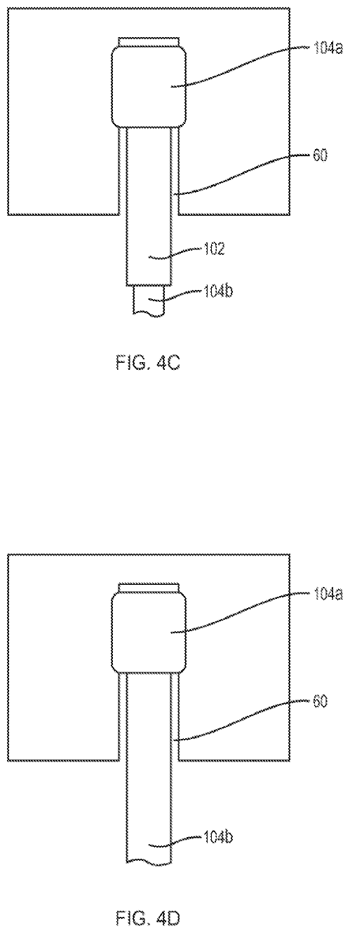

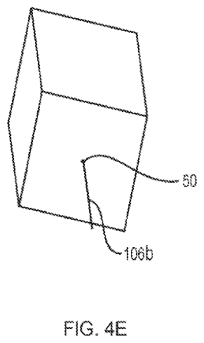

FIGS. 4A-E illustrate the use of the device 100 in a surgical repair, such as an anterior shoulder dislocation. However, the disclosure contemplates that the device 100 could also be used in any suitable repair that employs suture anchors. As shown in FIG. 4A, the user may first drill a hole 60 in bone, such as the glenoid 40. Then, using the tube 102 as a delivery device as shown in FIG. 4B, the user may insert the device 100 into the hole 60. As shown in FIG. 4C, the user may then deploy the device 100 such that the expanded distal end 104a of the first flexible member 104 forms an interference fit with the hole 60. As shown in FIG. 4D, the user may then withdraw the tube 102 from the hole 60, leaving the proximal end 106b of the second flexible member 106 extending from the hole 60 and ready to be used to complete the repair (FIG. 4E).

In examples not shown, the disclosure also contemplates the use of an outer or driver tube in the device 100. The disclosure also contemplates the use of more than two flexible members 104, 106. Furthermore, the disclosure contemplates that, rather than pulling the second flexible member 106 into the lumen 105 of the first flexible member 104, the second flexible member 106 could be pulled alongside the first flexible member 104 within the tube 102. Finally, the disclosure contemplates that the described expansion mechanism could be paired with a knotless trap (not shown) to generate a reducing suture anchor.

While the disclosure particularly shows and describes preferred examples, those skilled in the art will understand that various changes in form and details may exist without departing from the spirit and scope of the present application as defined by the appended claims. The scope of this present application intends to cover such variations. As such, the foregoing description of examples of the present application does not intend to limit the full scope conveyed by the appended claims.

1. A suture/anchor and delivery device combination comprising:
a tube having a distal end and a proximal end, the tube defining a channel extending from the distal end to the proximal end:
a first flexible strand having a distal end and a proximal end, the first flexible strand extending through the channel of the tube such that the distal end of the first flexible strand extends from the distal end of the tube; and
a second flexible strand having a first region with a first diameter and a second region with a second diameter, the first diameter selected to be smaller than the second diameter:
wherein the first region of the second flexible strand extends through a lumen defined by the first flexible strand such that the second region is adjacent the distal end of the first flexible strand; and
wherein the second region of the second flexible strand is configured to be pulled into the lumen of the first flexible strand to expand a diameter of the first flexible strand.

2. The suture/anchor and delivery device combination of claim 1, wherein the tube comprises a rigid or semi-rigid material.

3. The suture/anchor and delivery device combination of claim 1, wherein the first flexible strand comprises a hollow monofilament.

4. The suture/anchor and delivery device combination of claim 1, wherein the second flexible strand comprises one of mono-filament, muti-filament, braid or mesh.

5. The suture/anchor and delivery device combination of claim 1, further comprising a transition region extending between the first region and the second region of the second flexible strand.

6. The suture/anchor and delivery device combination of claim 1, wherein the first flexible strand is configured to expand to about 0.5 mm.

7. The suture/anchor and delivery device combination of claim 1, wherein a first region of the second flexible strand comprises a 2-0 suture.

8. The suture/anchor and delivery device combination of claim 1, wherein a second region of the second flexible strand comprises a No. 2 suture.

9. The suture/anchor and delivery device combination of claim 1, wherein a size of the second region of the second flexible strand is selected such that the second flexible strand can slide through the lumen of the first flexible strand without substantial interference.

10. The suture/anchor and delivery device combination of claim 1, wherein a size of the first flexible strand is selected such that it forms a friction fit with the channel of the tube.

11. The suture/anchor and delivery device combination of claim 1, wherein the first flexible strand extends through the channel of the tube such that the proximal end of the first flexible strand extends from the proximal end of the tube.

12. The suture/anchor and delivery device combination of claim 1, wherein the first region of the second flexible strand extends through the lumen defined by the first flexible strand such that the first region extends from the proximal end of the first flexible strand.

13. A method of surgical repair comprising:
inserting a suture/anchor and delivery device combination into a hole in bone, the device comprising:
a tube having a distal end and a proximal end, the tube defining a channel extending from the distal end to the proximal end:
a first flexible strand having a distal end and a proximal end, the first flexible strand extending through the channel of the tube such that the distal end of the first flexible strand extends from the distal end of the tube; and
a second flexible strand having a first region with a first diameter and a second region with a second diameter, the first diameter selected to be smaller than the second diameter:
wherein the first region of the second flexible strand extends through a lumen defined by the first flexible strand such that the second region is adjacent the distal end of the first flexible strand; and
pulling the second region of the second flexible strand into the lumen of the first flexible strand to expand a diameter of the first flexible strand.

14. The method of claim 13, wherein the expanded diameter of the first flexible strand is larger than a diameter of the channel of the tube.

15. The method of claim 13, wherein the expanded diameter of the first flexible strand forms an interference fit with the hole.

16. The method of claim 13, further comprising drilling the hole in the bone.

17. The method of claim 13, wherein the bone is a glenoid.

18. The method of claim 13, further comprising, after expanding the diameter of the first flexible strand, removing the tube from the hole.

19. The method of claim 13, wherein the first flexible strand extends through the channel of the tube such that the proximal end of the first flexible strand extends from the proximal end of the tube.

20. The method of claim 13, wherein the first region of the second flexible strand extends through the lumen defined by the first flexible strand such that the first region extends from the proximal end of the first flexible strand.
